(19) United States
(12) Patent Application Publication
(10) Pub. No.: US 2008/0319197 A1
(43) Pub. Date: Dec. 25, 2008

(54) Crystalline Form of Remifentanil Hydrochloride

(76) Inventors: Brian K. Cheng, Chesterfield, MO (US); Gary A. Nichols, Wildwood, MO (US); Catherine E. Thomasson, Webster Groves, MO (US)

Correspondence Address: Mallinckrodt Inc., 675 McDonnell Boulevard, HAZELWOOD, MO, 63042, US

(21) Appl. No.: 12/161,057

(22) PCT Filed: Jan. 19, 2007
(86) PCT No.: PCT/US07/01554

§ 371 Date: Jul. 16, 2008

Related U.S. Application Data
(60) Provisional application No. 60/765,280, filed on Feb. 3, 2006.

(51) Int. Cl. C07D 211/56

(52) U.S. Cl. 546/244

(57) ABSTRACT

The present invention relates to a crystalline polymorphic form of remifentanil hydrochloride. The invention also describes methods of preparing a polymorphic form of remifentanil hydrochloride.

### BACKGROUND OF THE INVENTION

I. Field of the Invention

The present invention relates to a novel crystalline form of remifentanil hydrochloride and methods for making crystalline forms of remifentanil hydrochloride.

II. Background of the Invention

Remifentanil (1-piperidinepropanoic acid, 4-(methoxy-carbonyl)-4-((1-oxopropyl)phenylamino)-, methyl ester; CAS No. 132875-61-7) is a synthetic opiod. It has a molecular formula C₂₀H₂₈N₂O₅ and the following structural formula:

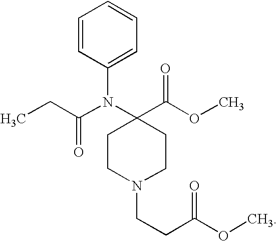

The most common salt of remifentanil is remifentanil hydrochloride (CAS No. 132539-07-2)

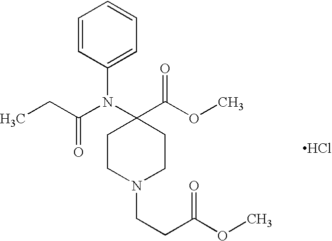

N-Phenyl-N-(4-piperidinyl)amides, such as remifentanil, and their preparation were originally described in U.S. Pat. No. 5,019,583 (the contents of which are incorporated in their entirety by reference). U.S. Pat. No. 5,466,700, the contents of which are incorporated in their entirety by reference, describe the use of the opioids described in U.S. Pat. No. 5,019,583 to induce and maintain anesthesia and conscious sedation. U.S. patent application Ser. No. 10/130,324, the contents of which are incorporated in their entirety by reference, describe pathways for the synthesis of fentanyl derivatives, including remifentanil.

Remifentanil is commercially available as an injection or an infusion under the brand name Ultiva® (GlaxoSmithKline). Its solid pharmaceutical form is a lyophilized powder for reconstitution for intravenous administration. Currently there exists no crystalline or polymorphic forms of remifentanil—remifentanil is only known to be amorphous. In addition, there exist no crystalline or polymorphic forms of any remifentanil salts. The present invention describes a crystal form of a remifentanil salt and a method for its production.

### SUMMARY OF THE INVENTION

One aspect of the invention is directed to a crystalline form of remifentanil hydrochloride.

A second aspect of the invention is directed to methods for preparing crystalline forms of remifentanil hydrochloride.

Other novel features and advantages of the present invention will become more apparent to those skilled in the art upon examination of the following or upon learning by practice of the invention.

### BRIEF DESCRIPTION OF THE FIGURES

FIG. 1 shows the pXRD pattern for remifentanil hydrochloride Form I.

FIG. 2 shows the pattern acquired for the Ultiva® sample (before grinding/heating) compared to reduced reference patterns for diglycine hydrochloride and various crystalline phases of glycine.

FIG. 3 shows a comparison of pXRD patterns for the Ultiva® sample (before and after grinding/heating) and crystalline remifentanil hydrochloride Form I.

FIG. 4 shows a DSC trace of remifentanil hydrochloride.

FIG. 5 shows a WVS trace of remifentanil hydrochloride.

### DETAILED DESCRIPTION OF THE INVENTION

The present invention describes a crystalline form of remifentanil hydrochloride and methods of making crystalline forms of remifentanil hydrochloride.

Commercially available remifentanil hydrochloride was found to be amorphous as demonstrated by pXRD. Using the following processes, crystalline forms of remifentanil hydrochloride were obtained. The crystalline character of the remifentanil hydrochloride was demonstrated by pXRD.

Crystalline forms of remifentanil hydrochloride may be prepared in accordance with the following general procedure.

Methyl 3-(4-anilino-4-carbomethoxy-piperidino) propionate is dissolved in a solvent. Any solvent can be used including acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethyl ether, diethylene glycol, diglyme, dimethylether, DMF, DMSO, dioxane, ethanol, ethyl acetate, ethylene glycol, glycerine, glyme, heptane, HMPA, HMPT, hexane, methanol, MTBE, nitromethane, pentane, petroleum ether, 1-propanol 2-propanol, pyridine, THF, water, o-xylene, m-xylene, and p-xylene. Preferably acetonitrile and chloroform are used as the solvent.

Once the methyl 3-(4-anilino-4-carbomethoxy-piperidino) propionate is dissolved in the solvent, an acyl donor is added. Preferably, acyl chlorides such as propionyl chloride is used as the acyl donor. The solution is stirred and heated. The temperature to which the solution is heated may depend on the solvent used and can range from about −25° C. to about 250° C. Preferably the solution is heated to a temperature of from about 40° C. to 80° C., more preferably from about 50° C. to about 70° C., most preferably about 60° C. The resulting solution is cooled and the remifentanil hydrochloride is allowed to crystallize out. The crystals are separated and analyzed. If necessary the crystals can be recrystallized. The recrystallization solvent may be the same as or different from the first crystallization solvent.

Specific non-limiting examples of processes are shown next merely for illustrative purposes.

Propionyl chloride (0.03 mL) was added to a stirring solution of methyl 3-(4-anilino-4-carbomethoxy-piperidino) propionate (1.5 g) in acetonitrile (10 mL). The solution was stirred at room temperature for 1 hour. Additional propionyl chloride (0.47 mL) was added and the solution was allowed to stir for another hour. The solution was heated to 60° C. for 2 hours then stirred at room temperature for approximately 48 hours. Precipitation occurred and the solvent was filtered off and the solid was washed with ethanol. This precipitate was determined to be remifentanil hydrochloride. Crystallization occurred in the mother liquor which contained acetonitrile and ethanol. The solvent was filtered off and the solid was washed with ethanol. This solid was found to be crystalline remifentanil hydrochloride. This solid was re-crystallized from isopropanol to obtain 99.19% pure remifentanil hydrochloride. The crystal was characterized by pXRD.

Remifentanil hydrochloride recovered from the mother liquor was neutralized with aqueous sodium bicarbonate solution and extracted into ethylacetate. The ethylacetate solution was dried over magnesium sulfate then concentrated in vacuo to obtain yellow oil. The oil was dissolved in acetonitrile (10 mL) to which propionyl chloride (0.3 mL) was added. The solution was heated to 60° C. overnight, cooled to room temperature, and then filtered to obtain a white powder, which was washed with acetonitrile.

The mother liquor was concentrated under vacuum to obtain a second oil. The second oil was dissolved in isopropanol (10 mL) to which concentrated hydrochloric acid (1 mL) was added. The resulting solution was dried under vacuum to obtain a light brown solid. Sufficient isopropanol was added to disperse the solid then filtered to obtain a white solid. The white solid was also found to be remifentanil hydrochloride and its crystal structure was characterized by pXRD.

### EXPERIMENTAL INFORMATION

Crystallization Experiments

For the slow evaporation experiments, each solvent/solvent system was saturated/near saturated with remifentanil hydrochloride in a small vial, and set aside at room temperature in a nitrogen purged desiccator. Following crystal growth, the solid material was, in some cases, filtered from the residual solvent using a fritted disc funnel.

Rapid evaporation experiments were performed by saturating/near saturating a particular solvent with remifentanil hydrochloride, and then evaporating off the solvent under a generous nitrogen purge.

Experiments described as “hot” were completed as follows. An aliquot of each solvent was saturated/near saturated with remifentanil hydrochloride at an elevated temperature. The solutions were then typically cooled in an ice bath. Following crystal growth, the solid material was filtered from the residual solvent using a fritted disc funnel.

Experiments in which two solvents were employed were accomplished using either a mixture of the two solvents, or by dissolving/suspending remifentanil hydrochloride in one solvent, and then adding the other solvent until the remifentanil hydrochloride was observed to completely dissolve. Solid material was at times filtered from the residual solvent using a fritted disc funnel.

Slurry Experiment

In a small vial (10 mL), remifentanil hydrochloride (48 mg) was slurried in isopropyl alcohol (0.5 mL) using a magnetic stir bar/plate. The slurry was analyzed by pXRD periodically to determine if any change in crystalline form had occurred.

Amorphous Preparation

A portion of remifentanil hydrorchloride (20 mg) was dissolved in MQ water (2.0 mL) in a vial (10 mL). The solution was then filtered into a 24/40 concentrator flask, and frozen using a dry-ice/acetone slush bath. The prepared sample was then lyophilized using a Savant—Freeze Dryer w/SpeedVac System—SS22.

Crystallization/Slurry/Amorphous Preparation Results

Remifentanil hydrochloride was crystallized from several different solvent systems, slurried for 23 days in isopropyl alcohol, and lyophilized. The solid material isolated from these crystallization experiments was characterized by at least one analytical technique.

DSC

A TA Instruments Q100—differential scanning calorimeter was used. The samples were weighed into a hermetic, aluminum pan and sealed with a pinhole lid. The samples were heated from 25° C. to 225° C. at a rate of 5° C. per minute (unless otherwise noted).

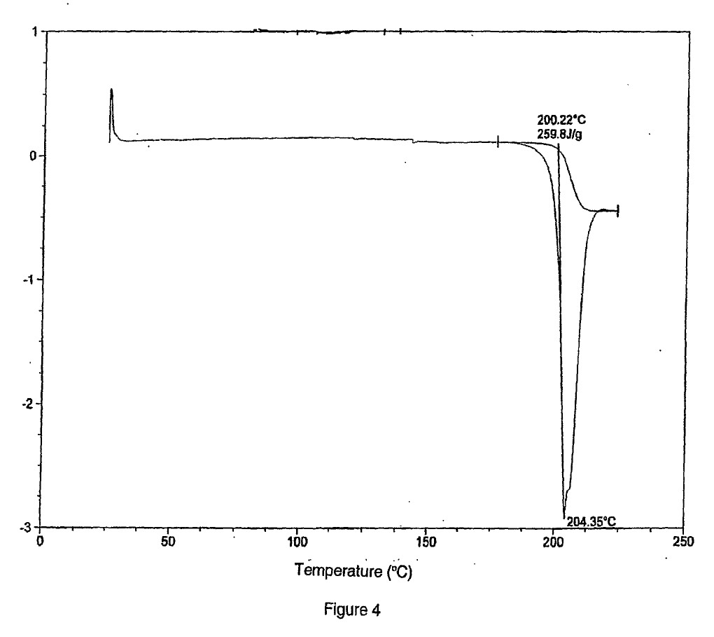

The DSC trace for crystalline remifentanil hydrochloride samples exhibited a large endothermic transition at approximately 200° C.—as shown in FIG. 4.

pXRD

A Siemens D500 X-ray Diffractometer was used. Each sample was uniformly crushed with a spatula edge, and placed on a quartz, zero-background holder. The following instrument parameters were utilized: Scan range—2.0 to 40.0 deg. 2θ, Step size—0.02 deg. 2θ, Scan time per step—1.0 seconds (2.0 seconds for the Ultiva® sample), Radiation source—copper Ka (1.5406 Å), X-ray tube power—40 kV/30 mA (45 kV/40 mA for the Ultiva® sample).

Single Crystal X-Ray Diffraction (SCXRD)

The two samples of remifentanil hydrochloride were used to obtain two single crystal X-ray diffraction structures for remifentanil hydrochloride. Powder X-ray diffraction patterns were simulated from these single crystal structures and compared to experimental patterns using the Materials Data software packages J-Powd & Jade.

X-Ray Powder Diffraction (pXRD) and Single Crystal X-Ray Diffraction (SCXRD) Results

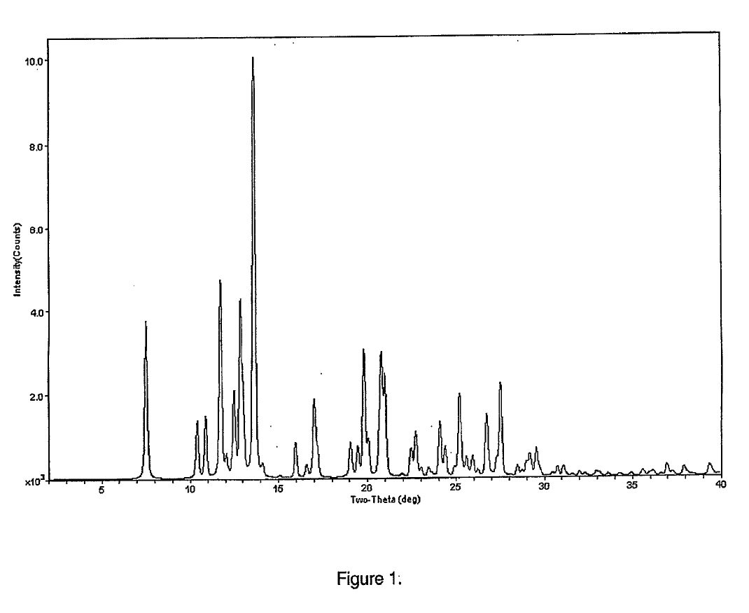

Two single crystal X-ray structure were solved as part of this study. Patterns simulated from these structures correspond to a single crystalline form of Remifentanil hydrochloride, designated remifentanil hydrochloride Form I (see FIG. 1). The following Table identifies peak values of Form I:

Peak Report for Remifentanil Hydrochloride - Form I

| Scattering Angle | |
|---|---|
| (degrees 2θ) | d-Spacing (Å) |
| *7.54 | 11.715 |
| *10.42 | 8.483 |
| *10.92 | 8.096 |
| *11.76 | 7.518 |
| 12.10 | 7.309 |
| *12.54 | 7.053 |
| *12.90 | 6.857 |
| *13.68 | 6.468 |
| 14.14 | 6.258 |
| 16.02 | 5.528 |
| 16.62 | 5.330 |
| *17.04 | 5.199 |
| 19.08 | 4.648 |
| 19.50 | 4.549 |
| *19.84 | 4.471 |
| *20.08 | 4.419 |
| *20.82 | 4.263 |
| *22.48 | 3.952 |
| *22.76 | 3.904 |
| *24.12 | 3.687 |
| 24.42 | 3.642 |
| *25.22 | 3.528 |
| 25.60 | 3.477 |
| 25.96 | 3.429 |
| *26.76 | 3.329 |
| *27.56 | 3.234 |
| 29.20 | 3.056 |
| 29.60 | 3.016 |

Crystalline remifentanil hydrochloride forms having at least five of the preceding peaks that are indicated by an asterix (+/−0.2 deg 2θ) are preferred embodiments of the invention. More preferable are forms having at least eight of the preceding peaks that are indicated by an asterix (+/−0.2 deg 2θ). Even more preferable are forms having at least twelve of the preceding peaks that are indicated by an asterix (+1-0.2 deg 2θ). Most preferably, the forms have all of the preceding peaks that are indicated by an asterix (+1-0.2 deg 2θ).

All of the remifentanil hydrochloride samples provided similar experimental pXRD patterns. All of these experimental patterns are reasonably similar to the simulated patterns obtained from the single crystal X-ray structures. In other words, all of the samples characterized are comprised of predominately one crystalline form, i.e., remifentanil hydrochloride Form I (see FIG. 1).

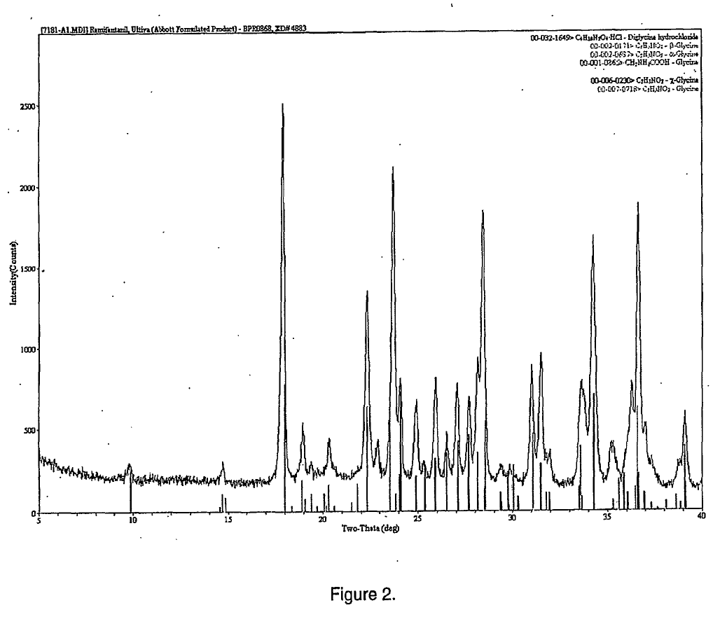

The Ultiva® sample (before grinding/heating) exhibited a pXRD pattern containing peaks/reflections consistent with the presence of crystalline glycine (possibly multiple phases) and diglycine hydrochloride (see FIG. 2).

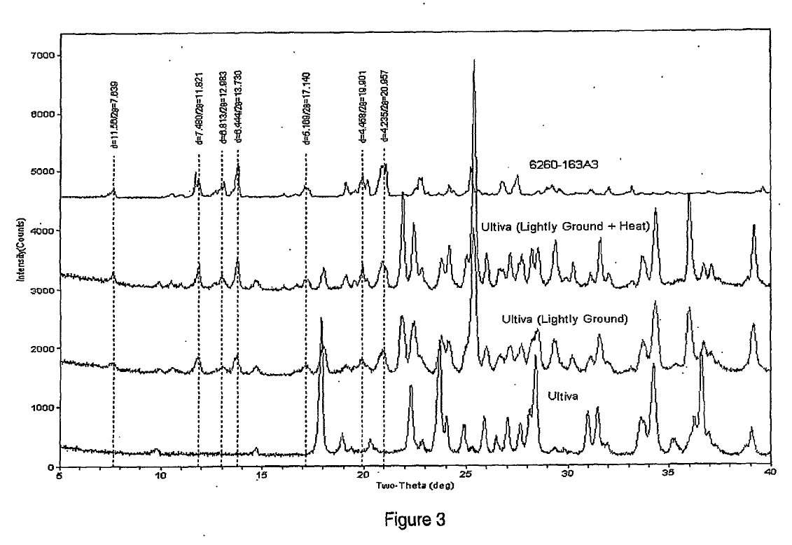

After the Ultiva® sample was lightly ground and/or heated on the pXRD plate, additional peaks/reflections were observed. Many of these newly observed reflections appear to be related to the presence of remifentanil hydrochloride Form I (see FIG. 3). FIG. 3 shows that the patterns for the ground/heat exposed Ultiva® and crystalline remifentanil hydrochloride share several common peaks (identified with dotted lines), which are not present in the original Ultiva® pattern.

The remifentanil hydrochloride (amorphous preparation) prepared via lyophilization exhibited a pXRD pattern consistent with that of amorphous material (no sharp peaks reflections).

The remifentanil hydrochloride slurried in isopropyl alcohol exhibited the same pXRD pattern for Form I during the 23-day period, and thus the crystalline form of the sample remained unchanged.

WVS Experiment

A VTI SGA-100 Water Vapor Sorption Balance was used. A portion of the second remifentanil hydrochloride sample was weighed into a platinum pan, and enclosed in the sample chamber. The three consecutive adsorption/desorption isotherms were acquired under isothermal conditions, 25° C.

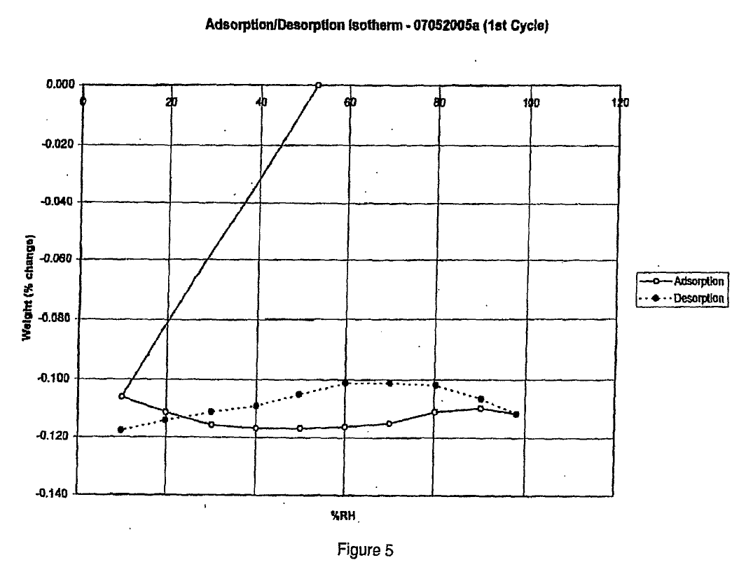

A portion of the second remifentanil hydrochloride sample was subjected to several consecutive adsorption/desorption cycles (10-98% RH). The sample did not adsorb a significant amount of water (<0.1% by mass) during any of the adsorption cycles. In addition, the pXRD pattern of the second remifentanil hydrochloride sample following the WVS experiment remained unchanged—see FIG. 5.

Optical/Hot-Stage Microscopy

An Olympus BX61 microscope equipped with an INSTEC STC200 hot-stage was utilized for the described analyses. The sample was viewed using a Sony 3CCD Color Video Camera.

A small amount of each sample was dispersed onto a glass slide, and placed into the hot-stage. Samples were heated from room temperature to 225° C. at a rate of 5° C. per minute, while being observed under the microscope at a magnification of 200×.

The first remifentanil hydrochloride sample, comprised of small block-like/tablet-like chunks of crystalline (birefringent) material, exhibited no drastic changes prior to melting at temperatures above 190° C.

We claim:

1. A crystalline form of remifentanil hydrochloride, characterized by an X-ray diffraction pattern having at least five of the following peaks: 7.54, 10.42, 10.92, 11.76, 12.54, 12.90, 13.68, 17.04, 19.84, 20.08, 20.82, 22.48, 22.76, 24.12, 25.22, 26.76, and 27.56+/−0.2 degrees 2θ.

2. The crystalline form of remifentanil hydrochloride of claim 1, characterized by an X-ray diffraction pattern having at least eight of the following peaks: 7.54, 10.42, 10.92, 11.76, 12.54, 12.90, 13.68, 17.04, 19.84, 20.08, 20.82, 22.48, 22.76, 24.12, 25.22, 26.76, and 27.56+/−0.2 degrees 2θ.

3. The crystalline form of remifentanil hydrochloride of claim 1, characterized by an X-ray diffraction pattern having at least twelve of the following peaks: 7.54, 10.42, 10.92, 11.76, 12.54, 12.90, 13.68, 17.04, 19.84, 20.08, 20.82, 22.48, 22.76, 24.12, 25.22, 26.76, and 27.56+/−0.2 degrees 2θ.

4. The crystalline form of remifentanil hydrochloride of claim 1, characterized by an X-ray diffraction pattern having the following peaks: 7.54, 10.42, 10.92, 11.76, 12.54, 12.90, 13.68, 17.04, 19.84, 20.08, 20.82, 22.48, 22.76, 24.12, 25.22, 26.76, and 27.56+/−0.2 degrees 2θ.

5. The crystalline form of remifentanil hydrochloride of claim, 1 characterized by an x-ray diffraction pattern substantially as shown in FIG. 1.

6. The crystalline form of remifentanil hydrochloride of claim 1, characterized by a differential scanning calorimetry thermogram taken at a heating rate of 5° C./min. in a closed pan that exhibits a large endothermic transition at approximately 200° C.

7. A crystalline form of remifentanil hydrochloride, characterized by an X-ray diffraction pattern having peaks at about 7.5, 11.7, 12.9, 13.7, 19.8, 20.8, 25.2, and 27.6+/−0.2 degrees 2θ.

8. A method for preparing at least one crystalline form of remifentanil hydrochloride, the method comprising:
a) combining an acyl chloride and methyl 3-(4-anilino-4-carbomethoxy-piperidino) propionate in a solvent;
b) heating the resulting solution; and
c) cooling the solution to allow the remifentanil hydrochloride to crystallize from the mother liquor.

9. The method according to claim 8, wherein the acyl chloride is propionyl chloride.

10. The method according to claim 8, wherein the solvent is acetonitrile.

11. The method according to claim 8, wherein the solution is stirred at room temperature before heating.

12. The method according to claim 8, further comprising:
d) re-crystallizing the remifentanil hydrochloride from a second solvent.

13. The method according to claim 12, wherein the second solvent is isopropanol.

14. The method according to claim 12, wherein the remifentanil hydrochloride is at least about 99% pure.

15. The method according to claim 8, further comprising recovering additional remifentanil hydrochloride from the mother liquor.
